(19) United States
(12) Patent Application Publication
(10) Pub. No.: US 2017/0127959 A1
(43) Pub. Date: May 11, 2017

(54) ANIMAL VITAL SIGN DETECTION SYSTEM

(71) Applicant: KONINKLIJKE PHILIPS N.V., Eindhoven (NL)

(72) Inventors: Elvira Johanna Maria Paulussen, Eindhoven (NL); Giuseppe Coppola, Eindhoven (NL); Petrus Johannes Marie de Lange, Veldhoven (NL); Alphonsus Tarcisius Jozef Maria Schipper, Strampoy (NL)

(21) Appl. No.: 15/318,421

(22) PCT Filed: Jun. 15, 2015
(86) PCT No.: PCT/EP2015/063252

§ 371 Date: Dec. 13, 2016

(30) Foreign Application Priority Data
Jun. 27, 2014 (EP) 14174646.1

(51) Int. Cl. A61B 5/024; F21V 8/00; A01K 29/00

(52) U.S. Cl. CPC A61B 5/02427; A01K 29/005; G02B 6/0001; A61B 2090/306

(57) ABSTRACT

An animal vital signs detection system is provided which comprises at least one photoplethysmography (PPG) sensor (100) having at least one light source (110), at least one photo sensor (120) and at least one light guide unit (130) which is adapted to guide light from the light source (110) through hairs, fur or pelt to a skin of an animal (100) and light from the skin through hair, fur or pelt of the animal (1000) to the at least one photo sensor (120). By providing the light guide coupled to the light source (110) and the photo sensor (120), the PPG sensor (100) can be placed even on hair, fur or pelt of an animal (1000) while still being able to effectively direct light to the skin and detect light from the skin of an animal (1000). The ratio between a length and a diameter of the light guides is >4.

### FIELD OF THE INVENTION

The invention relates to an animal vital sign detection system as well as a method for detecting vital signs of an animal.

### BACKGROUND OF THE INVENTION

It is well-known that the heart rate of a user or an animal can be monitored by optical sensors like by photoplethysmography (PPG) sensors. These sensors measure the variation in the blood volume in human or animal tissue and can determine a pulse signal of the heartbeat of a human or an animal.

US 2003/0166996 A1 discloses a method and an apparatus for measuring biological conditions of animals by acquiring and analyzing their biological signals. These biological signals can be a photoplethysmogram.

WO 91/15151 A1 discloses a perinatal pulse oximetry probe. The probe comprises a light source and a light detector. Furthermore, the probe comprises a cluster of light-transmissive bumps covering the light source or the light detector.

WO 96/41566 discloses a sensor for optical blood oximetry. The sensor comprises two light emitters positioned in close proximity to each other emitting light at different wavelengths and a light detector. The sensor furthermore comprises light emitter terminals consisting of a bundle of optical fibres.

US 2012/0004517 discloses a pulse oximeter system for animal research. The oximeter comprises a light source and a light receiver. The oximeter is placed at a tail of a rat.

WO 2004/075746 A2 discloses a method of measuring physiological parameters to obtain a pulse oximetry signal.

WO 2008/058328 A1 discloses a system for non-invasive monitoring of respiratory parameters in sleep disordered breathing.

### SUMMARY OF THE INVENTION

It is an object of the present invention to provide an animal vital signs detection system as well as a corresponding method which is able to detect vital signs of animals in a more reliable and effective way even in the presence of hair, fur or pelt.

In an aspect of the present invention, an animal vital signs detection system is provided that comprises at least one PPG sensor having a housing, at least one light source, at least one photo sensor and at least one light guide unit which is protruding from the housing and adapted to guide light from the light source through hairs, fur or pelt to a skin of an animal and light from the skin through hair, fur or pelt of the animal to the at least one photo sensor. By providing the light guide coupled to the light source and the photo sensor, the PPG sensor can be placed even on hair, fur or pelt of an animal while still being able to effectively direct light to the skin and detect light from the skin of an animal. The light guide unit comprises at least one second light guide having a first and second end and being adapted to guide light from the light source through hair, fur or pelt to the skin of an animal and at least one first light guide having a first and second end and being adapted to guide light from the skin through hairs, fur or pelt of the animal to the at least one photo sensor. The light guides comprise a length and a diameter. The ratio between the length and the diameter is >4. Accordingly, separate light guides are provided for light from the source to the skin and for light from the skin to the sensor. Due to the ratio (length—diameter), the light guides can effectively penetrate the hair, fur or pelt of an animal and can be placed in contact with the skin of an animal.

According to a further aspect of the invention, the first light guide comprises a plurality of first light guide arms which are commonly coupled at the first or second end (i.e. the entrance or exit surface/area) of the first light guide. The second light guide unit can comprise a plurality of second light guide arms which are commonly coupled at the first or second end of the second light guide. By providing a plurality of light guide arms, the area of the skin towards light is projected or from which light is detected can be increased thus increasing the reliability of the detection.

According to a further aspect of the invention, a cross section or diameter of the first end of the first and second light guide is smaller than a cross section or diameter of the second ends of the first and second light guides. In other words, the cross section is increasing towards the direction of the light source or the photo sensor such that all the light is directed onto the skin or onto the photo sensor.

According to a further aspect of the invention, the PPG sensor can be operated in a transmittance mode or in a reflectance mode. In the transmittance mode, the at least one light source can emit light at a wavelength between 850 nm and 950 nm. In the reflectance mode, the at least one light source can emit light at a wavelength between 500 nm and 600 nm.

In a further aspect of the invention, in the transmittance mode, the at least one light source can emit light at a wavelength of 660 nm and 905 nm.

According to a further aspect of the invention, the animal vital signs detection system furthermore comprises a processing unit adapted to process the output of the at least one PPG sensor. The processing unit furthermore comprises a cardio-respiratory failure detection unit adapted to detect a cardio-respiratory failure of an animal based on the output of the PPG sensor. Thus the cardio-respiratory action of animals can be effectively monitored.

According to a further aspect of the invention, the processing unit comprises a heart rate detecting unit adapted to detect a heart rate based on the output of the PPG sensor and to compare the detected heart rate with a threshold value and to output an alert if the detected heart rate exceeds the threshold value. By means of the heart rate detection unit, the animal vital signs detection system can be used together with detection animals which are able to detect specific substances like explosive, drugs, blood, etc. If the detection animal detects any one of these substances, its heart rate will increase and the increased heart rate will be detected by the animal vital signs detection system and an alert is outputted.

The invention also relates to the use of at least one PPG sensor having at least one light source and at least one photo sensor for detecting vital signs of an animal. The at least one PPG sensor is arranged at least at one position on an animal including at least one of a nose, an ear, a tail, a head or neck, a thigh-bone or leg and a hoof. Preferably, the PPG sensor is arranged at these positions because at these positions, either a large artery is located just beneath the skin or the skin is thin at these positions and contains vessels such that the PPG sensor can detect a heart rate. Preferably, at a maxillary artery which is supplying blood to the face of a horse, the PPG sensor can be placed. In addition, the PPG sensor can be attached in the region of a radial artery. Furthermore, the sensor can be arranged in the region of a digital artery.

An in particular advantageous position for a PPG sensor is the upper inner part of the tail, e.g. of a horse.

The invention also relates to a use of at least one PPG sensor having at least one light source and at least one photo sensor for detecting vital signs of an animal. The at least one PPG sensor is operable in a transmittance mode or a reflectance mode. In the transmittance mode, the at least one light source emits light at a wavelength between 850 and 950 nm. In the reflectance mode, the at least one light source emits light at a wavelength between 500 and 600 nm.

According to an aspect of the invention, the PPG sensor arranged at the tail, the head or neck, the thigh-bone or leg or the hoof of the animal is operated in the reflectance mode. A PPG sensor arranged at the nose or ear of an animal is operated in the transmittance mode.

According to an aspect of the invention, the PPG sensor unit further comprises a light guide unit having at least one first light guide having a first and second end and being adapted to guide light from the skin through hairs, fur or pelt of the animal to the at least one sensor and at least one second light guide having a first and second end and being adapted to guide light from the light source through hair, fur or pelt to a skin of an animal.

The invention also relates to a method of detecting animal vital signs with at least one PPG sensor having at least one light source and at least one photo sensor. The at least one PPG sensor is arranged to at least one position on an animal including a nose, an ear, a tail, a head or neck, a thigh-bone or leg and a hoof.

The measurements of vital signs of animals like the heart rate is advantageous as it enables for example for horses a more effective training method as the training activities can be adjusted in their level of intensity depending on the detected heart rate. Furthermore, it should be noted that exercise, physical condition, environmental temperature, disease, excitement and age can influence the heart rate of an animal. Raised heart rates may indicate severe dehydration, colic, shock, infection, advanced heart and lung disease and septicemia. On the other hand, reduced heart rates may suggest low body temperature, heart disease, pressure on the brain or a possible pre-terminal state with an impending collapse of circulation.

A PPG sensor is an optical sensor which is used to obtain a plethysmogram which is a volumic measurement of an organ. The PPG sensor illuminates the skin and measures changes in the light absorption. The PPG sensor is able to detect the change in volume caused by the pressure pulse. In particular, the amount of light which is transmitted or reflected is measured by a photo sensor.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows a block diagram of an animal vital signs detection system according to an aspect of the invention,

FIG. 2A-2E each show a schematic representation of an animal, in particular a horse, with at least one animal vital signs detection sensor,

FIG. 3 shows a schematic representation of a further animal, in particular a cow, with at least one animal vital signs detection sensor according to an aspect of the invention,

FIG. 4A-4D each show a schematic representation of an animal vital signs detection sensor according to an aspect of the invention, and

FIG. 5A and 5B each show a schematic representation of an animal vital signs detection sensor according to an aspect of the invention.

### DETAILED DESCRIPTION OF EMBODIMENTS

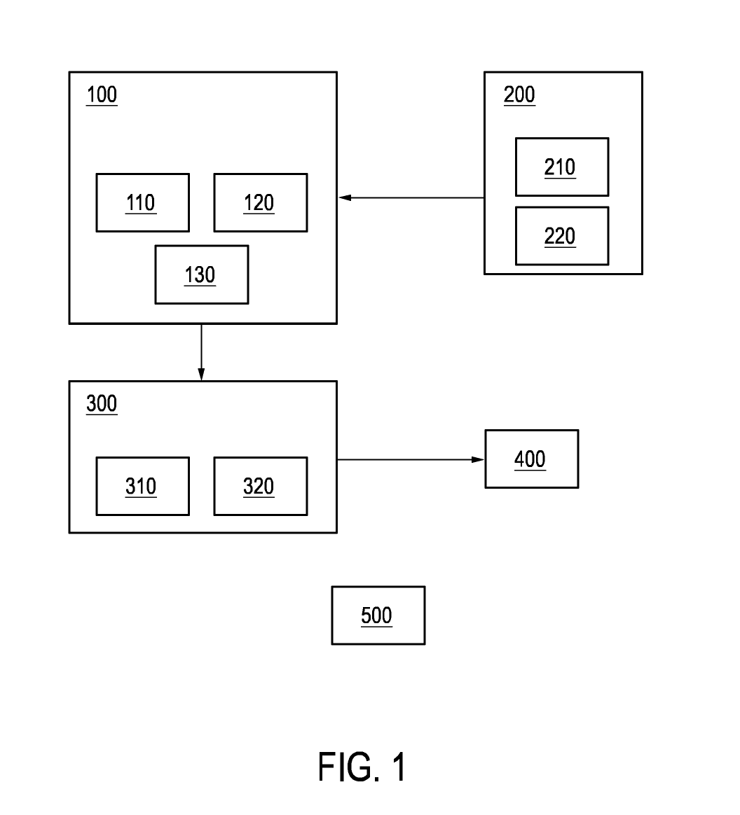

FIG. 1 shows a block diagram of an animal vital signs detection system according to the invention. The animal vital signs detecting system according to an aspect of the invention comprises at least one PPG sensor 100, optionally a control unit 200 for controlling the operation of the sensor 100, a processing unit 300 for determining vital signs from the output of the at least one sensor 100 and optionally an output unit for outputting the vital signs as determined by the processing unit 300. The animal vital signs detection system according to an aspect of the invention may also comprise at least one fastening unit 500 for fastening at least the sensor 100 to parts of an animal 1000, 1100, 1200. The output unit 400 can be provided to output the detected vital signs wirelessly or based on wires. The output unit 400 may optionally also comprise a display for displaying the detected vital signs.

The PPG sensor 100 comprises at least one light source 110 for emitting light and at least one photodetector 120 for detecting light from the light source. The PPG sensor 100 optically measures the variation in blood volume in human or animal tissue and can detect a pulse signal from these measurements. The light source 110 can be implemented as light emitting diodes (LEDs). The wavelength of these diodes can be according to an aspect of the invention between 520 nm and 850 nm. The PPG sensor 100 can be implemented as a transmitting sensor or as a reflectance sensor measuring the reflections from the light emitted by the light source 110. In other words, the PPG sensor 100 can be operated in a transmission mode or in a reflectance mode. In the transmission mode, the PPG sensor 100 can emit light at a wavelength between 650-850 nm. In the reflection mode, the light sources 110 can emit light with wavelengths between 520 and 570 nm. It should be noted that the path length of a reflection in an animal tissue is shorter than the path length of a transmittance of light in the animal tissue. It should be noted that the signal-to-noise ratio of the heartbeat is based on an amount of absorption of the light emitted by the light sources 110 in the blood of the animal.

According to the invention, transmittance measurements are more robust than reflectance measurements. Accordingly, the transmittance measurements, i.e. the PPG sensor 100 operated in the transmittance mode, requires less power than in the reflectance mode. It should be noted that an optical path length through the tissue of an animal in the transmittance mode is larger than in the reflectance mode. Accordingly, the signal-to-noise ratio of the measured variation in the transmittance mode is larger as in the reflectance mode. Furthermore, in the transmittance mode, longer wavelengths are used which can penetrate into deeper regions of the skin and may have less interaction with scattered particles in the tissue of the animal.

In the reflectance mode, the PPG sensor 100 emits light at wavelengths which have a high absorption by hemoglobin, for example at peak wavelength between 520 and 570 nm. In the transmittance mode, the PPG sensor 100 allows more sensitive measurements as the travelling distance of the radiation via the skin is larger and thus the amount of hemoglobin which can absorb the radiation from the light sources can be larger. In particular, the wavelengths of the light used by the PPG sensor 100 in the transmittance mode are in the red or infrared region as they allow a larger penetration depth through the skin and have a low scattering effect at high wavelength. In the transmittance mode, the PPG sensor 100 can use a wavelength at approx. 660 nm and at approx. 905 nm. It should be noted that these two wavelengths enable different absorption rates by the blood. These absorption rates are dependent on the blood oxy-hemoglobin level. If the blood is oxygenated, the red light at 660 nm is absorbed to a reduced amount as compared to the absorption of IR light. If the blood is deoxygenated, the red light is absorbed to a greater extent than the IR light.

According to an aspect of the invention, the wavelengths as used by the sensor 100 can be between 850 and 950 nm for the transmittance mode. These wavelengths are advantageous as it enables measurements through the hair, fur or pelt of animals.

When the animal vital sign detection system according to an aspect of the invention is to be used, care must be taken to ensure that the light emitted by the light source 110 of the PPG sensor 100 actually reaches the skin of the animal through the hairs, fur or pelt and can also penetrate the skin of the animal. Furthermore, care should be taken that the reflected or transmitted light can also reach the photo sensor 120 of the PPG sensor 100, i.e. care must be taken that the light can also penetrate the fur, pelt or hair of the animal on its way towards the photo sensor 120. This can be achieved by using at least one light guide 130 between the skin or tissue 1000 of an animal and the light source 110 or the photo detector 120. The light source and the photo detector can be arranged inside a housing of the animal vital signs detector while the light guide protrudes from the housing. The light guides have a length (extending or protruding from the housing) and a diameter or cross section. The ratio between the length and the diameter is >4 to enable an effective penetration of the hair or fur of an animal.

According to an aspect of the invention, in the transmittance mode, the PPG sensor 110 uses a wavelength between 850 and 950 nm. In addition or alternatively, several different wavelengths can be used by the light source 110 in the transmittance mode. According to an aspect of the invention, the wavelengths can be 660 nm and 905 nm. The usage of these two wavelengths is advantageous as light with these two wavelengths is absorbed differently by the blood in accordance to the blood oxy-hemoglobin level.

In the reflectance mode, the PPG sensor 100 can use wavelengths between 500 and 600 nm and in particular between 520 and 570 nm. These ranges are advantageous as the absorption by the hemoglobin in the blood is relatively high. In addition or alternatively, in the reflectance mode, the PPG sensor 100 can use wavelengths above 850 nm, in particular between 850 and 950 nm. At these wavelengths, the hemoglobin absorption is relatively high while the pelt, fur or hair absorption is relatively low. In addition or alternatively, several wavelengths like a first wavelength between 520 and 570 nm and a second wavelength at 905 nm can be used. The usage of multiple wavelengths is advantageous as the absorption rate of blood dependant on the blood oxy-hemoglobin level is different. It should be noted that if the blood is oxygenated, the red light is absorbed to a less extent than when using IR light. On the other hand, if the blood is deoxygenated, the red light is absorbed to a greater extent than the IR light.

The control unit 200 can optionally be able to control the PPG sensor 100 in a transmittance mode or in a reflectance mode. Optionally, the control unit 200 can comprise a transmittance unit 210 for controlling the PPG sensor in the transmittance mode and/or a reflectance unit 220 for controlling the PPG sensor 100 in a reflectance mode.

Optionally, the processing unit 300 may also be part of the PPG sensor 100. Alternatively, the processing unit 300 may be implemented as a dedicated unit. Optionally, the processing unit 300 can comprise a heart rate detecting unit 310 for detecting or determining the heart rate of an animal based on the measurements from the PPG sensor 100.

Optionally, the processing unit 300 can comprise a cardio-respiratory failure detection unit 320 for detecting a cardio-respiratory failure of animals. Here, the vital signs as detected by the PPG sensor 100 or the heart rate as determined by the heart rate detection unit 310 are monitored. It is well known that parameters like the heart rate or oxygen saturation can be an indication of an illness of animals. If the vital signs of an animal can be monitored by means of the PPG sensor 100, this would be advantageous as preemptive measurements can be taken in order to avoid an illness of an animal or in order to avoid the worsening of the illness.

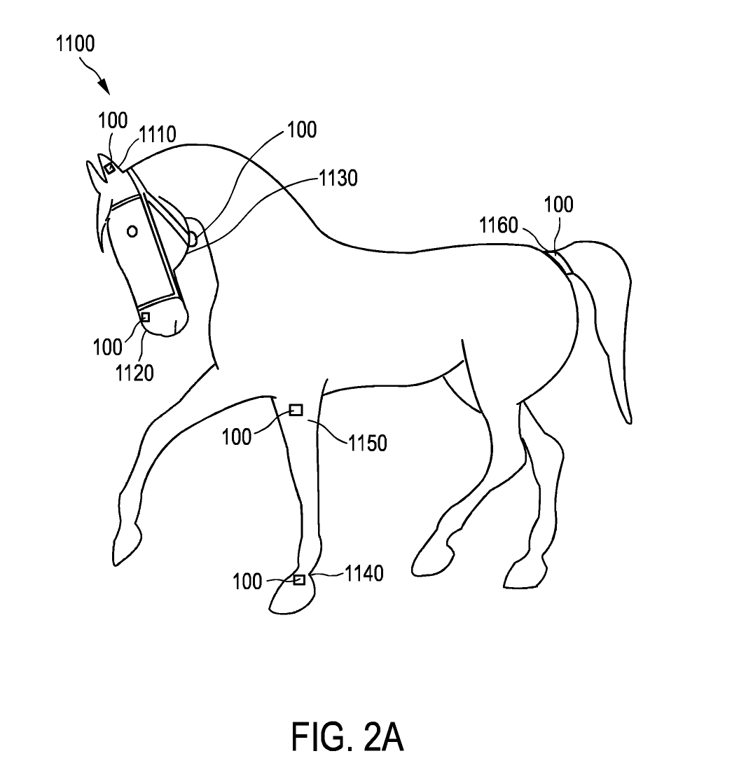

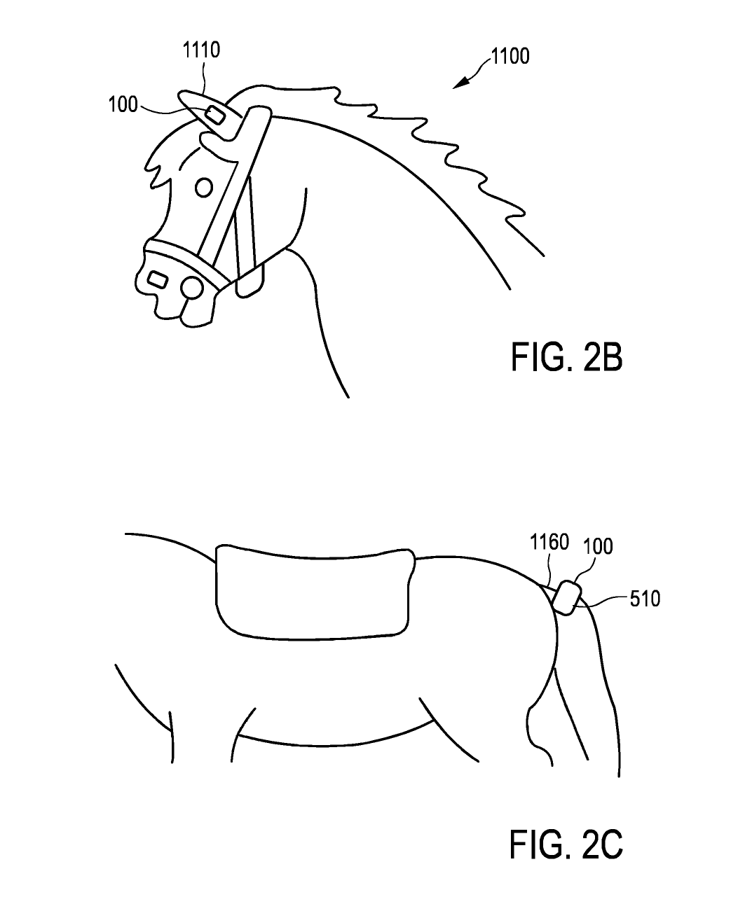

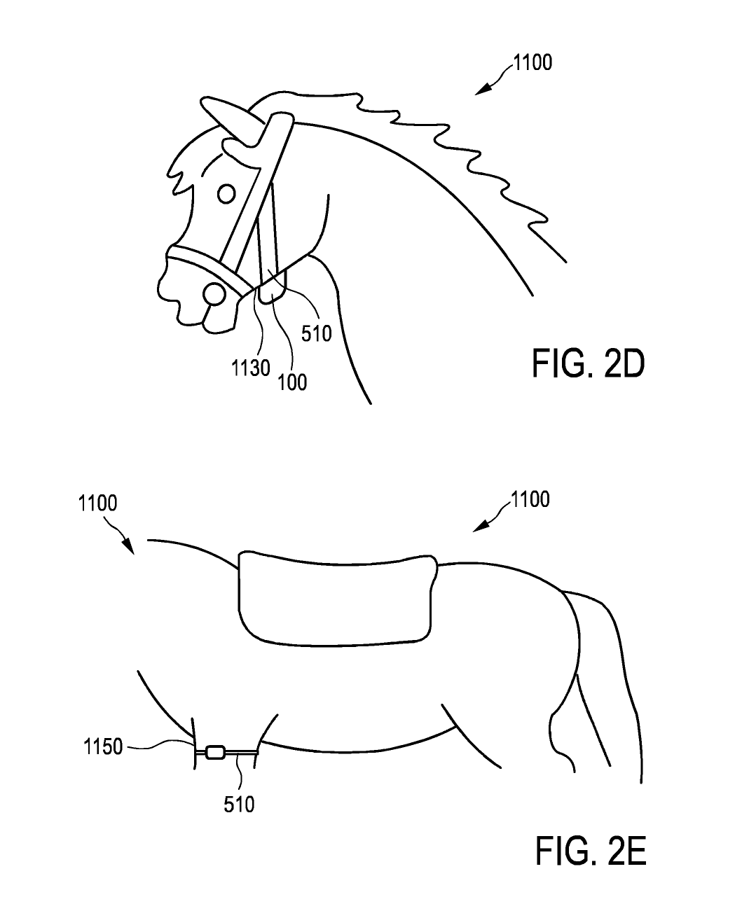

According to an aspect of the invention, if the PPG sensor 100 is attached to the hoof of an animal, for example as shown in FIG. 2A, the measurements of the sensors can be used to detect any premature laminitis.

Preferably, the PPG sensor 100 is embodied as a small sensor (e.g. the ends of the light guide units can be ≦1 mm; the light source and/or the photo sensor can be e.g. 1 mm such that the PPG sensor unit can be e.g. 5 mm) and can be attached to the ear of the animal for example as shown in FIG. 2A and FIG. 2B to detect vital signs of the animal.

According to an aspect of the invention, the heart rate detection unit 310 can also be used to monitor the heart rate of an animal to which a PPG sensor 100 is attached. This can for example be used for detection animals like detection dogs which are trained to sense explosives, drugs, bloods or missing humans. If such a detection animal has sensed explosive, drugs, blood or missing humans, typically the heart rate will increase. Such an increased heart rate can be detected by means of the heart rate detecting unit 310 and an alarm signal can be outputted via an output unit 400.

It should be noted that also dolphins can be used as detecting animals. In fact, dolphins are nowadays used by the military to detect mines in the water. According to an aspect of the invention, the output unit 400 may comprise a wireless transmitter for transmitting an alert signal wirelessly.

The usage of the heart rate detecting unit 110 with detection animals is advantageous as some animals are not able to indicate that they have sensed or detected an explosive, drug, blood or the like. Furthermore, the detection animals do not need to visibly indicate anymore that they have found the respective substance as the handler will know that the substance has been found as the heart rate of the detection animal is increased. Accordingly, for example a detection dog will not have to bark any longer to indicate that he has found a certain substance. In case of ferrets or special birds, the usage of a PPG sensor 100 and the heart rate detection unit 310 is advantageous as only the heart rate needs to be monitored to indicate an easy and reliable method that the animal has detected some of the substances.

Optionally, the animal vital signs detecting system may also comprise a position sensor like a GPS sensor. The data from the position sensor may be associated to the output signals of the sensor 100. In addition, a time stamp may also be included or associated to the output signal from the PPG sensors 100. This can be advantageous as the data from the PPG sensors 100 can be analyzed afterwards.

FIG. 2A-2E each shows a schematic representation of an animal, in particular a horse, with at least one animal vital signs detection sensor. In FIG. 2A, an animal 1000, in particular a horse 1100, is depicted. Furthermore, several possible positions for the PPG sensor 100 according to an aspect of the invention are disclosed. Preferably, the PPG sensor 100 is arranged at spots where large arteries are located just beneath the skin. According to an aspect of the invention, a PPG sensor 100 can be attached to an ear 1110 or to a nose 1120 (in particular the ala of the nose or the wing of the nose). A PPG sensor 100 can also be arranged at the tail 1160 of a horse 1100, in particular around the top at an inner side of the tail. Furthermore, a PPG sensor 100 can be attached to the head or neck 1130 of a horse 1100. Moreover, a PPG sensor 100 can be arranged at the thigh-bone or leg 1150 of the horse. In addition, the PPG sensor 100 can be arranged at the hoof coronet 1140. These positions are all advantageous as they enable a good detection of vital signs of an animal by means of a PPG sensor 100 according to an aspect of the invention.

According to FIG. 2B, the PPG sensor 100 can be attached to the ear or nose (the ala of the horse). These two spots are in particular advantageous as the skin is thin and contains a plurality of vessels. The PPG sensors 100 attached to the ear 1100 or the nose 1120 of the horse are in particular used for PPG sensor 100 operated in the transmittance mode as less hair or pelt is present. It should be noted that here a slab of skin is present where the light source can be placed at one side and the photo sensor can be placed on the other side to measure the transmission.

The PPG sensors 100 arranged at the tail 1160 (FIG. 2C), the head or neck 1130 (FIG. 2D), the thigh-bone 1150 (FIG. 2E) or the hoof coronet 1140 can in particular be used in the reflectance mode.

As disclosed in FIG. 2C, the PPG sensor 100 can be arranged at the tail and in particular at the inside of the base of the tail, where no hairs are present. This position is advantageous as the sensor can be attached to the base of the tail by means of a flexible band 510, such that the sensor can be attached in an unobtrusive manner.

While at the ear 1110 or the nose 1120 of the horse, pelt or hair is not present, at the other mentioned positions, hair or pelt can be present. Accordingly, here, the reflectance mode of the PPG sensor is used. According to an aspect of the invention, the PPG sensor 100 can be pressed against the skin of the animal for example by means of a flexible band 510. The flexible band 510 can therefore be part of the fastening device 500. Preferably, the PPG sensor 100 is pressed against the skin of the animal. Optionally, the PPG sensor 100 may comprise a light guide unit 130 for guiding light from the light source to the skin or for guiding light from the skin to the photo sensor 120. These light guides 130 can be used at those positions where hair, fur or pelt is present.

Optionally, those parts of the animal where the PPG sensor is to be attached can be shaved before attaching the PPG sensor 100.

Optionally, the PPG sensor and in particular the light source 110 and the photo sensor 120 are smaller than 5 mm.

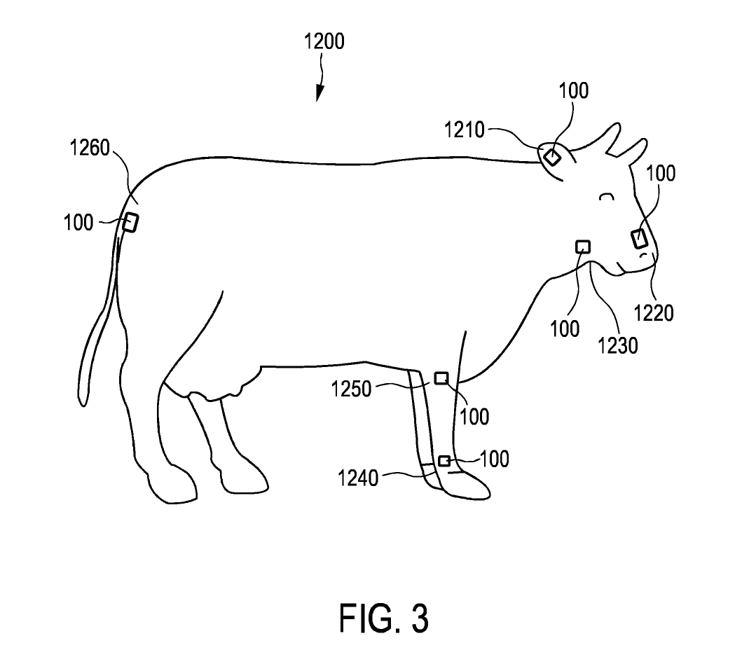

FIG. 3 shows a schematic representation of a further animal, in particular a cow, with at least one animal vital signs detection sensor according to an aspect of the invention. In FIG. 3, in particular a cow 1200 is depicted. Furthermore, several positions for the PPG sensor 100 according to the invention are depicted. The PPG sensors can be applied to the same spots as described above with reference to FIG. 2. Therefore, a PPG sensor 100 according to an aspect of the invention can be attached to the ear 1210, the nose 1220, the neck or head 1230, the hoof or hoof coronet 1240, the leg 1250 and/or the upper (inner) part of the tail 1260. The ear 1210 and the nose 1220 can be used as position for detection in the transmittance mode while the other positions can be used for a detection in the reflectance mode.

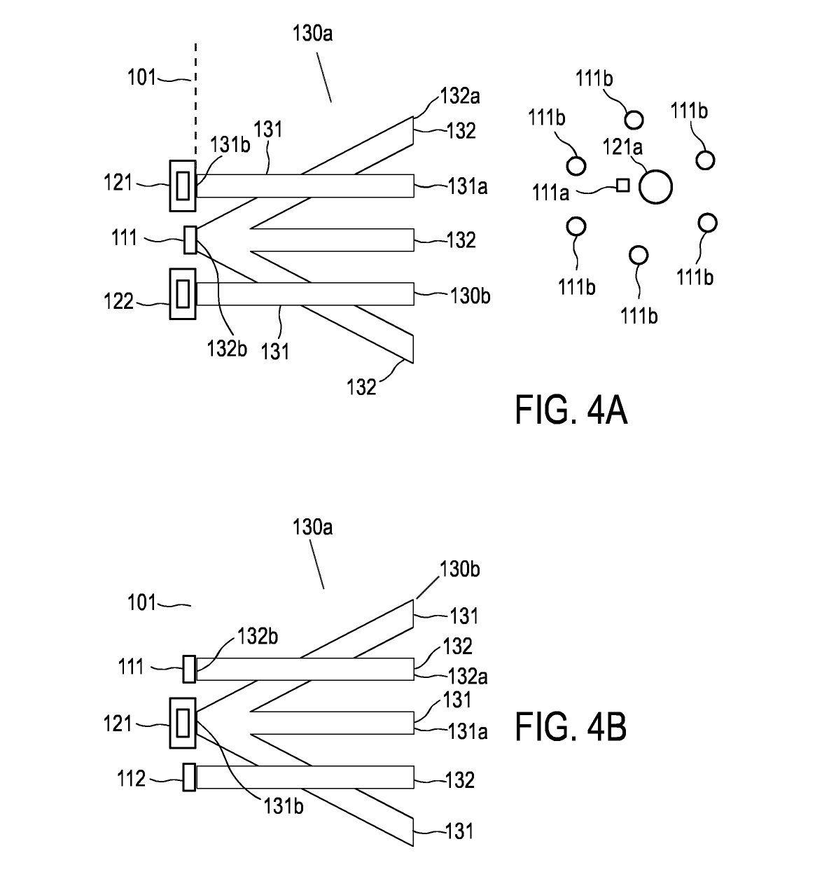

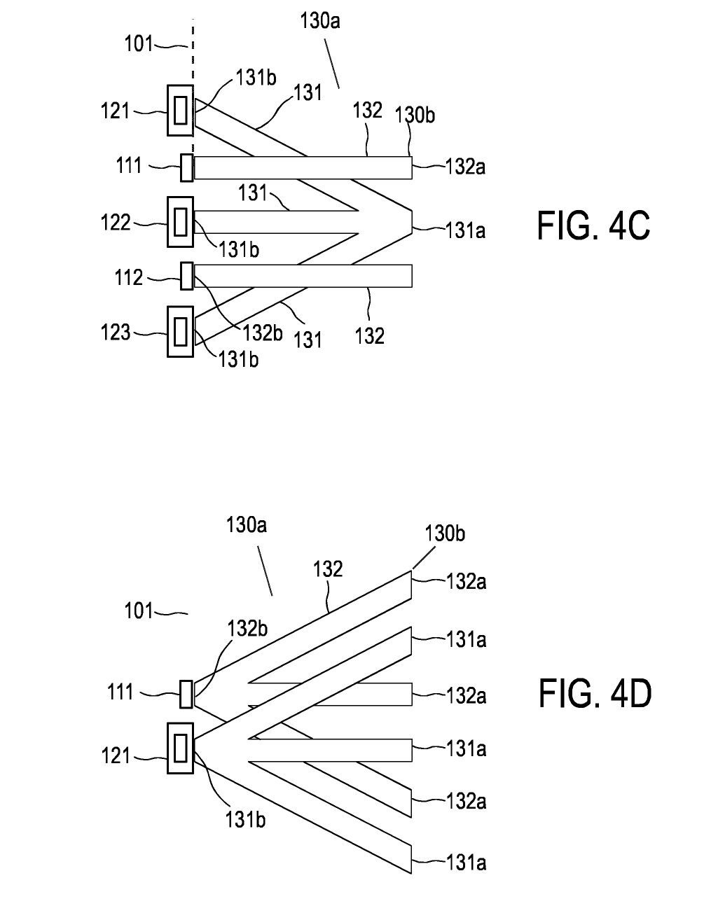

FIG. 4A shows a schematic representation of an animal vital signs detection sensor according to an aspect of the invention. In order to effectively be able to transmit light through pelt or hair of animals to the skin and to effectively receive any transmitted or reflected light, the PPG sensor 100 comprises a housing 101, at least one light guide 130 for guiding light from the light source 110 towards the skin of an animal and/or for guiding light from the skin of the animal towards the photo sensor 120. The light guide protrudes from the housing 101. Based on the detected light from the skin of the animal, the PPG sensor and/or the processing unit 300 can determine vital signs like heart rate, heart rate variability HRV, respiration rate, oxygen saturation, energy expenditures, etc. In FIG. 4A, an aspect of the invention is depicted where a PPG sensor 100 comprises a housing 101, one LED light source 111 and two photo sensors (photo diodes) 121, 122. A first light guide 131 is coupled to each of the two photo diodes 121, 122. The first light guide 131 comprises a first end 131a and a second end 131b, wherein the second end 131b is attached to the photo diode 121, 122. Accordingly, the first end 131a can be placed against the skin of an animal. Optionally, the second ends 131a, 132a of the first and second light guides can be flat such that they can be placed efficiently against the skin of an animal. Alternatively, the second ends 131a, 132a can have a lens to focus the light onto the skin or tissue 1000. The second light guides 132 are coupled to the LED 111. In particular, the second light guide 132 comprises a plurality of arms, e.g. at least three arms (or alternatively six arms), wherein the second ends (132b) of these arms are coupled together with the LED light 111, while the first ends 132a can be placed against the skin of an animal. Accordingly, with a single LED 111, three or more spots of light 111b can be achieved on the skin of an animal by means of the light guide arms 131, 132. Accordingly, with the arrangement of the second light guides 132, the effective covering range of the light from the LED 111 can be multiplied.

On the right hand side of FIG. 4A, a light spot pattern on the skin of the animal when using the PPG sensor according to FIG. 4A is depicted. Here, a middle arm 132 of the light guide 132 creates a light spot 111a. The other arms of the second light guide 132 create light spots 111b. The position and diameter of the light spots will depend on the arrangement of the light guide arms. The spot 121a depicts the spot where the photo sensor 121 together with its light guide 131 detects light coming out from the skin of the animal or light being reflected by the skin of the animal. The light guides 131, 132 have a length 130a and a diameter 130b.

In FIG. 4B, a PPG sensor 100 according to different aspects of the invention is disclosed. Here, a PPG sensor with a housing 101 and two LEDs 111, 112 are disclosed. However, one or a plurality of LEDs is also possible. Two second light guide arms 132 protrude from the housing 101 and are coupled with their second ends 132b to the first and second LED 111, 112 and are used to direct light from the LEDs 111, 112 towards the skin of an animal. The arrangement of the first light guides 131 can correspond to the arrangement of the second light guides as described according to FIG. 4A. Accordingly, at least one light guide, e.g. three to six light guides 131 can be provided, wherein the second ends of the light guides 131 can be commonly coupled to the photo diode 121 while the first ends 131a can be pressed against the skin of an animal. It should be noted that the number of light guides or light guide arms can be selected according to the specific design and can therefore be one or more than one. The light guides 131, 132 have a length 130a and a diameter 130b.

In FIG. 4C, a further aspect of a PPG sensor according to an aspect of the invention is depicted. Here, the PPG sensor comprises a housing 101 and three photo diodes 121, 122, 123 and two LEDs 111, 112 are depicted. Second light guides 132 are coupled to the first and second LED 111, 112. The first light guide is split into different light guides, wherein the second ends 131b of the light guides are coupled to the first, second or third photo diode 121, 122, 123, while the first ends 131a are commonly coupled together to form a single contact point towards the skin of the animal. The first and second light guides 131, 132 protrude from the housing 101. The light guides 131, 132 have a length 130a and a diameter 130b.

The advantage of this embodiment maybe looks contradictory, but this makes it possible to detect a larger area of the skin and as the viewing angle of the photo sensor is limited, if multiple photo sensors are used, etendu is preserved and all the light can be captured.

In FIG. 4D, the PPG sensor 100 is depicted with a single LED 111 and a single photo diode 121. Multiple light guides 132 protrude from the housing 101 and are commonly coupled to the LED 111 as well as multiple light guides are commonly coupled to the photo diode 121.

The light guides according to the invention can also be embodied as protrusions or thin pipe, wherein a small printed circuit board PCB comprises the LED and the photo sensor and is placed at one end (entrance or exit) of the small pipe. Or, in other words, instead of the light guides according to FIG. 4A 4D, small pipes can be provided to guide light through the hair, fur or pelt.

The light guide can comprise optical transparent material. The material is selected according to the wavelength of the LEDs as used. Alternatively or additionally, the light guides can be embodied as hollow light guides which are coated with a reflective material which is in particular reflecting light with the used wavelength. According to a further aspect of the invention, the light guides can consist of an optical transparent material and can be coated with a reflective material at their inside.

The light guides according to the invention have a diameter or width 130b of <1 mm. The length 130a of the light guides (i.e. the distance the light guides protrude from the housing 101 of the PPG sensor) can be several mm or several tens of mm. According to an aspect of the invention, the ratio between the length of the light guides and the diameter or width of the light guides is >4. According to an aspect of the invention, the ratio can also be 10 or 20. The ratio of the length 130a of the light guides in relation to the diameter or width 130b of the light guides is important if the PPG sensors are used for animals due to the presence of hair or fur. By means of the light guides according to an aspect of the invention, the light guides can easily penetrate the fur or hair of the animals and be in contact with the tissue or skin of the animals.

If the light guides are implemented as hollow light guides for example in form of hollow light pipes, this leads to a reduced cross talk between adjacent light guides. The light guide unit 130 has a length 130a and the light guides have a diameter 130b. As can be seen from the FIGS. 4A to 4D, a length of a first light guide may be different to a length of a second light guide. Optionally, the diameter of a first light guide may be different from the diameter of a second light guide.

The present invention is not limited to horses or cows but is applicable to mammals like alpaca, bisons, camels, dogs, donkeys, goats, lama, mules, pigs, sheep, yak, etc. The animal vital signs detection sensor according to the invention can for example also be used with dolphins Furthermore, the PPG sensor according to the invention can also be used for life stock like horses, cows, pigs, etc. Furthermore, the PPG sensor according to an aspect of the invention can be used for any farm animals. Optionally, the PPG sensor according to the invention may also be used for birds, dogs, cats, etc.

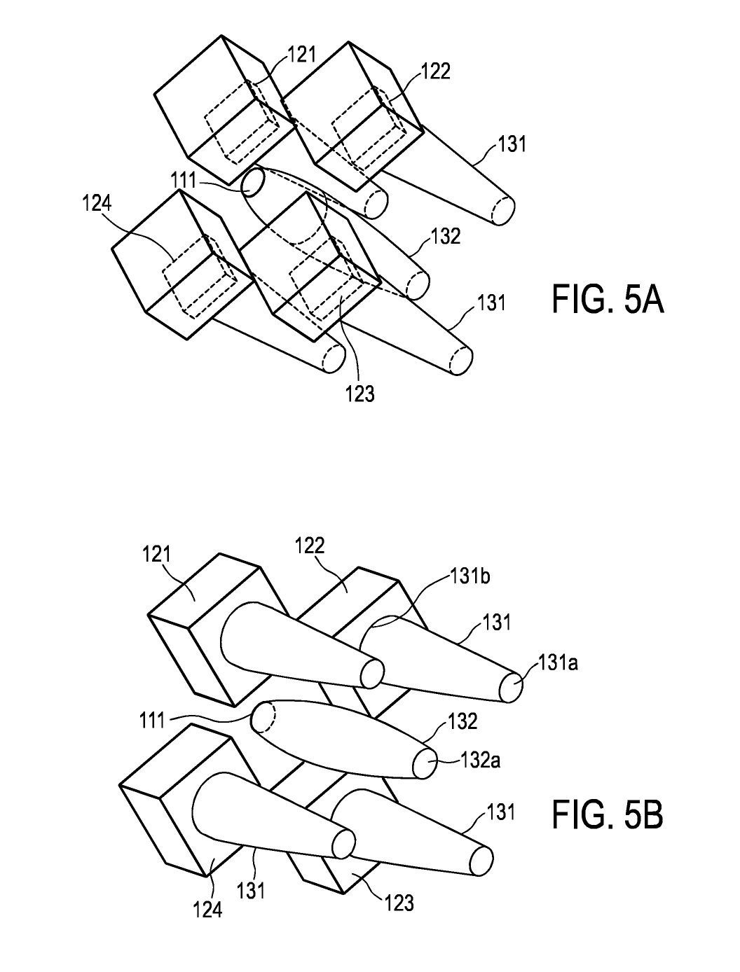

FIG. 5A and 5B each show schematic representations of an animal vital sign detection sensor according to an aspect of the invention. Here, the PPG sensor 100 comprises a single LED 111 to which a second light guide 132 is coupled. Furthermore, the PPG sensor comprises four photo diodes 121, 122, 123 and 124 which are each coupled to a first light guide 131. The light of the LED 111 is directed towards the skin of an animal via the second light guide 132. At or in the skin of the animal, the light is scattered and reflected or transmitted light is guided through each of the first light guides 131 towards one of the photo diodes 121 124, where the light is detected.

FIG. 5B shows a representation of the animal vital signs detector sensor according to FIG. 5A. Accordingly, the PPG sensor comprises four photo diodes 121 124 each with a first light guide 131 as well as a LED 111 with a second light guide 132.

According to this aspect of the invention, the diameter of the light guides at their distal ends is smaller than the diameter at the end which is coupled to the photo sensor or the LED 111.

According to an aspect of the invention, a photoplethysmography sensor 100 is provided which comprises at least one light source 110, at least one photo sensor 120 and at least one guide unit 130, which is adapted to guide light from the light source 110 through hairs, fur or pelt to a skin of an animal 1000 and light from the skin through hairs, fur or pelt of the animal 1000 to the at least one photo sensor 120.

The PPG sensor 100 can be attached to a snaffle or bridle for a horse. The sensor can in particular be attached to a strap at the throat of the horse to detect vital signs of the horse.

According to a further aspect of the invention, the PPG sensor can be attached to several positions as indicated above for example by means of flexible straps. Preferably, a PPG sensor 100 is attached to the upper inner part of the tail for example of a horse.

It should be described that the above described animal vital sign detection system and a method for detecting vital signs of an animal are only used in non-therapeutic applications to measure or detect vital signs of animals.

In the above, the PPG sensor 100 has been described as being operable in a transmittance mode and in a reflectance mode. It should be noted that according to an aspect of the invention, also a PPG sensor 100 is provided which is only able to operate in the transmittance mode. Furthermore, according to an aspect of the invention, the PPG sensor may also only be able to operate in the reflectance mode. Even though the PPG sensor 100 is only able to operate either in the transmittance mode or in the reflectance mode, the above described principles are still valid and such a PPG sensor can be used according to the invention.

According to an aspect of the invention, the LED can be replaced by OLED or laser units.

According to an aspect of the invention, the wavelength of the light source can also be between 600-60 nm, e.g. 620 nm.

Other variations of the disclosed embodiment can be understood and effected by those skilled in the art in practicing the claimed invention from a study of the drawings, the disclosure and the appended claims.

In the claims, the word “comprising” does not exclude other elements or steps and in the indefinite article “a” or “an” does not exclude a plurality.

A single unit or device may fulfill the functions of several items recited in the claims. The mere fact that certain measures are recited in mutual different dependent claims does not indicate that a combination of these measurements cannot be used to advantage. A computer program may be stored/distributed on a suitable medium such as an optical storage medium or a solid state medium, supplied together with or as a part of other hardware, but may also be distributed in other forms such as via the internet or other wired or wireless telecommunication systems.

Any reference signs in the claims should not be construed as limiting the scope.

1. Animal vital signs detection system, comprising at least one photoplethysmography sensor having a housing, at least one light source, at least one photo sensor and at least one light guide unite, which is adapted to guide light from the light source through hairs, fur or pelt to a skin of the animal and light from the skin through hairs, fur or pelt of the animal to the at least one photo sensor,
wherein the at least one light source and the at least one photo sensor are arranged in the housing,
wherein the light guide unit comprises at least one second light guide having a first end and a second end coupled to the at least one light source and being adapted to guide light from the light source through hairs, fur or pelt to the skin of the animal and at least one first light guide having a first end adapted to be placed directly onto the skin of the animal and a second end coupled to the at least one photo sensor and being adapted to guide light from the skin through hairs, fur or pelt of the animal to the at least one photo sensor
wherein the at least one first and second light guide protrudes from the housing with a length and a diameter,
wherein a ratio between the length and the diameter is >4.

2. Animal vital signs detection system according to claim 1, wherein the first light guide comprises a plurality of first light guide arms which are commonly coupled at the first or second end of the first light guide, and/or wherein the second light guide comprises a plurality of second light guide arms which are commonly coupled at the first or second end of the second light guide.

3. Animal vital signs detection system according to claim 2, wherein a diameter of the first ends of the first and second light guides is smaller than a diameter of the second ends of the first and second light guides, or wherein a diameter of the second ends of the first and second light guides is smaller than a diameter of the first ends.

4. Animal vital signs detection system according to claim 3, wherein the at least one photoplethysmography sensor is operable in a transmittance mode or a reflectance mode, wherein in the transmittance mode the at least one light source emits light at a wavelength of between 850 nm and 950 nm, and wherein in the reflectance mode the at least one light source emits light at a wavelength of between 500 nm and 600 nm.

5. Animal vital signs detection system according to claim 4, wherein, in the transmittance mode, the at least one light source emits light at a wavelength of 660 nm and 905 nm, and, in the reflectance mode, the at least one light source emits light at a wavelength of 660 nm and 905 nm.

6. Animal vital signs detection system according to claim 4, further comprising a processing unit adapted to process an output of the at least one photoplethysmography sensor wherein the processing unit comprises a cardio-respiratory failure detection unit adapted to detect a cardio-respiratory failure of an animal based on the output of the photoplethysmography sensor.

7. Animal vital signs detection system according to claim 6, wherein the processing unit comprises a heart rate detection unit adapted to detect a heart rate based on the output of the photoplethysmography sensor, to compare the detected heart rate with a threshold value and to output an alert if the detected heart rate exceeds the threshold value.

8. Animal vital signs detection system according to claim 1, wherein the diameter of the light guides is <1 mm.

9. Method of detecting animal vital signs with at least one photoplethysmography sensor having a housing, at least one light source and at least one photo sensor, wherein the at least one light source and the at least one photo sensor are arranged in the housing, wherein the at least one first and second light guide protrudes from the housing with a length and a diameter,
wherein a ratio between the length and the diameter is >4, said photoplethysmography sensor detecting vital signs of the animal comprising the steps of:
arranging the at least one photoplethysmography sensor at at least one position on an animal including at least one of a nose, an ear, a neck, a thigh-bone or leg and a hoof, and
detecting vital signs of the animal by means of said photoplethysmography sensor.

10. Method according to claim 9, wherein the at least one photoplethysmography sensor arranged at a neck, thigh-bone or leg or a hoof of the animal is operated in a reflectance mode.
